(19) United States
(12) Patent Application Publication
(10) Pub. No.: US 2006/0228384 A1
(43) Pub. Date: Oct. 12, 2006

(54) Control of biofilm with a biofilm inhibitor

(75) Inventor: Gary R. Eldridge, Encinitas, CA (US)

Correspondence Address: THOMPSON COBURN, LLP, ONE US BANK PLAZA, SUITE 3500, ST LOUIS, MO, 63101, US

(73) Assignee: Sequoia Sciences, Inc.

(21) Appl. No.: 11/099,800

(22) Filed: Apr. 6, 2005

(51) Int. Cl. A61K 31/7034; A61K 31/202; A61K 31/496; A61K 31/65; A61K 31/43; A61K 31/545; A61K 31/4745

(52) U.S. Cl. 424/400; 514/35; 514/154; 514/200; 514/192; 514/291; 514/253.08; 514/312; 514/560; 514/210.09

(57) ABSTRACT

The present invention provides methods for preventing, reducing, inhibiting or removing bacterial biofilm. The present invention also provides methods for controlling acne and chronic bacterial infections. The present invention further provides a method for identifying agents that prevent, reduce, inhibit or remove bacterial biofilm.

### GOVERNMENTAL SUPPORT

Part of this work was supported by the U.S. National Institutes of Health under the grant no. R42 RR016363-02. The United States Government has certain rights in this invention.

### FIELD OF THE INVENTION

The present invention generally relates to methods and compounds useful for inhibiting, reducing, preventing, or removing biofilms produced by microorganisms. In particular, the invention provides a method for the control of biofilms using palmitoleic acid or its derivative as a biofilm inhibiting agent.

### BACKGROUND

Bacterial biofilms exist in natural, medical, and engineering environments. Biofilms offer a selective advantage to a microorganism to ensure its survival, or allow it a certain amount of time to exist in a dormant state until suitable growth conditions arise. Unfortunately, this selective advantage poses serious threats to animal health, especially human health. For example, biofilms are involved in 65% of human bacterial infections. Biofilms are involved in prostatitis, biliary tract infections, and urinary tract infections, cystitis, lung infections, sinus infections, ear infections, acne, rosacea, open wounds, and chronic wounds.

Chronic bacterial infections are serious medical problems in the United States. In chronic bacterial infections, biofilms protect bacteria from antibiotics and the host's immune response mechanisms, thus increasing the rates of recurring symptoms and resistance to antibiotics. Examples of antibiotics are Macrobid for the treatment of acute UTI (cystitis) and Tobramycin for the treatment of lung infections of cystic fibrosis patients. In addition to cystitis and cystic fibrosis, chronic bacterial infections include, but are not limited to dental caries, periodontitis, otitis media, and nosocomial infections due to contact lens and central venous catheters.

Biofilm inhibitors will have a substantial medical impact by treating many chronic infections, reducing catheter- and medical device-related infections, and treating lung and ear infections. The potential market for potent biofilm inhibitors is exemplified by the sheer number of cases in which biofilms cause medical problems. The biofilm inhibitors may be used to control microorganisms existing in extracellularly or intracellularly of living tissues. They may be used to cure, treat, or prevent a variety of conditions, such as, but are not limited to, arterial damage, gastritis, urinary tract infections, otitis media, leprosy, tuberculosis, benign prostatic hyperplasia, chronic prostatitis, chronic lung infections of humans with cystic fibrosis, osteomyelitis, bloodstream infections, skin infections, open or chronic wound infections, and any other acute or chronic infection that involves or possesses a biofilm.

Cystic fibrosis patients have chronic lung infections that are currently treated with the antibiotic tobramycin. The principal organism found in the lungs of cystic fibrosis patients is Pseudomonas aeroginosa, existing within a biofilm. Presently, current antibiotics, specifically antibacterial compounds, cannot provide effective treatment for biofilms of chronic infections, because antibiotic therapy fails to eradicate the biofilm. The Cystic Fibrosis Foundation estimated that approximately 30,000 patients have cystic fibrosis in the United States. These patients desperately need new medications that prevent the formation of biofilms in their lungs. Research supports the conclusion that therapeutics that interfere with the formation of biofilms might assist in the treatment of these patients.

As treatments for patients with gastritis, biofilm inhibitors can be used to prevent the attachment of Helicobactor pylori to gastric epithelial cells preventing their eventual invasion into these cells or inhibiting or reducing subsequent virulence factors, which can result in inflammation. Biofilm inhibitors can also be used to prevent or reduce the risks associated with the virulence factors of H. pylori, for example, by reducing arterial damage, which can lead to an increased risk of stroke. As treatments for urinary tract infections, biofilm inhibitors can be used to prevent, control, reduce, or eradicate the population of E. Coli that reside intracellularly in bladder cells, which resist conventional antibiotics and evade a host's immune systems.

Each year in the United States, over 7 million patients receive medical device implants, which include central venous catheters (CVC), endotracheal tubes, mechanical heart valves, pacemakers, and prosthetic joints. Approximately one-half of these patients develop nosocomial infections, and approximately 80,000 deaths per year are attributed to nosocomial infections.

According to the guidelines of the Center for Disease Control (CDC), the level of CVC bacterial colonization should be determined by the semi-quantitative or quantitative method (Centers for Disease Control and Prevention, “Guidelines for the Prevention of Intravascular Catheter-Related Infections”, MMWR. 51:RR-10, 2002). In these guidelines, a positive blood culture and more than 10³ colony-forming units (CFU) per catheter segment, as measured by the quantitative method, suggest that the CVC is the cause of the bloodstream infection (BSI). These guidelines have been used in peer-reviewed studies that examine nosocomial BSI. The results of these studies suggest a direct relationship between the probability of obtaining a BSI and the CFU per catheter segment. Therefore, reducing the bacterial colonization of a catheter should lower the incidence of nosocomial BSI.

Clinical studies show that the use of antiseptic-coated or antibiotic-impregnated polyurethane catheters significantly reduces bacterial colonization of CVC and nosocomial BSI. A study in 1997 showed that in comparison to untreated catheters, the use of polyurethane catheters coated with chlorhexidine and silver sulfadiazine resulted in statistically significant reductions in the level of catheter colonization and the incidence of BSI (Maki, D. G. et. al., “Prevention of Central Venous Catheter-Related Bloodstream Infection by Use of an Antiseptic-Impregnated Catheter”, Ann. Int. Med. 127(4):257-266, 1997). In a peer-reviewed clinical study, the superiority of the antibiotic-impregnated catheters versus the anti-septic impregnated catheters was demonstrated (Darouiche, R. et. al., “A Comparison of Two Antimicrobial-Impregnated Central Venous Catheters”, New Engl. Jour. Med. 340(1): 1-8, 1999). In 2003, a study in which polyurethane catheters impregnated with minocycline and rifampin were used in both medical and surgical intensive care units demonstrated BSI reductions of 8.3 to 3.5 and 4.8 to 1.3 per 1,000 days of inpatient hospitalization, respectively (Hanna, H. A., et. al., “Antibiotic-Impregnated Catheters Associated with Significant Decrease in Nosocomial and Multidrug-Resistant Bacteremias in Critically Ill Patients”, Chest. 124(3):1030-1038, 2003). This study also showed a statistically significant decrease in bacteremia caused by vancomycin-resistant Enterococci.

The results of these and other studies have led the CDC to strongly recommend the use of antimicrobial- or antiseptic-impregnated CVC in adult patients whose catheters are anticipated to remain in place for more than 5 days (Centers for Disease Control and Prevention, “Guidelines for the Prevention of Intravascular Catheter-Related Infections”, MMWR. 51:RR-10, 2002). Antimicrobial catheters have also been recommended for use in patients who have a high risk of developing nosocomial BSI, such as patients with burns or resistance to bacterial infections, patients undergoing cancer treatments, hemodialysis, or transplantation, and patients receiving emergency care. With a large number of patients falling into these high-risk categories, reducing nosocomial BSI would offer a significant societal benefit. Another approach for reducing BSI is to directly inhibit the bacterial mechanisms that form biofilms.

In the United States, the market for antibiotics is greater than $8.5 billion. After cardiovascular therapeutics, antibiotics are the second largest drug market in the United States. The size and growth of this market are continually fueled by an increased resistance to conventional antibacterial therapies, with approximately 70% of bacteria found in hospitals resisting at least one of the most commonly prescribed antibacterials. Biofilms are involved in 65% of human bacterial infections. Accordingly, biofilm inhibitors could significantly affect the antibiotic market.

Antibiotics traditionally have been discovered using the susceptibility test methods of the National Committee for Clinical Laboratory Standards (NCCLS), which identify compounds that specifically effect growth or the killing of bacteria. These methods involve inoculation of bacterial species in a suitable growth medium, followed by the addition of a test compound and plot of the bacterial growth over a time period post-incubation. These antibiotics are not effective therapeutics for chronic infections involving biofilms because these NCCLS methods do not test compounds in a preformed biofilm. Accordingly, the methods of the NCCLS used to identify current antibiotics do not apply to chronic infections involving biofilms.

When compounds are employed as biofilm inhibitors, they inhibit, reduce, prevent, or remove biofilms produced by microorganisms. The biofilm may offer a selective advantage to the microorganism to ensure its survival or allow it an undetermined amount of time to exist dormant until suitable growth conditions arise. The biofilm may directly or indirectly cause a disease or unfavorable health condition in a host. Biofilm inhibitors used to treat or prevent chronic infections from microorganisms or biofilm inhibitor-impregnated or coated medical devices offer numerous advantages.

Biofilm inhibitors have an alternative mechanism of action. Currently, successful treatment of nosocomial infections often requires an administration of a combination of products, such as amoxicillin/clavulanate and quinupristin/dalfopristin, or administration of two antibiotics simultaneously. The present invention provides an alternative approach to treating chronic infection; a subject is administered a combination of a biofilm inhibitor and antibiotic. The two compounds provide different mechanisms of action.

Bacteria have no known resistance to biofilm inhibitors. Minocycline and rifampin have been used widely to treat infections, and microbial resistance to these antibiotics is emerging. A study of an intensive care unit revealed that six out of seven vancomycin-resistant enterococci were resistant to rifampin (Hanna, H. A., et. al, “Antibiotic-Impregnated Catheters Associated with Significant Decrease in Nosocomial and Multidrug-Resistant Bacteremias in Critically Ill Patients”, Chest. 143(3):1030-1038). Biofilm inhibitors could extend the product life of current antibiotics. To date, doxycycline, an analog of minocycline, is a first-line therapy for inhalation and cutaneous anthrax. With the dramatically increased threat of bioterrorism, prevention of the emergence of resistance to this class of antibiotic is crucial. Recently, it has been reported that Bacillus anthracis are resistant to ciprofloxacin (Price, L. B., et. al., “In vitro selection and characterization of Bacillus anthracis mutants with high-level resistance to ciprofloxacin”, Antimicrob. Agents Chemother. 57:2362-2365, 2003). Clearly, microbes acquire antibiotic resistance quickly, challenging researchers to develop new antibiotics. Biofilm inhibitors provide an alternate strategy to control dangerous, and possibly lethal, bacterial infection. Biofilm inhibitors are not likely to trigger growth-resistance mechanisms or affect the growth of the normal human flora.

Biofilm inhibitors act specifically on the biological mechanisms that provide bacteria protection from antibiotics and a host's immune system. In one study of urinary catheters, rifampin was unable to eradicate methicillin-resistant Staphylococcus aureus in a biofilm but was effective against planktonic, or suspended cells (Jones, S. M., et. al., “Effect of vancomycin and rifampicin on methicillin-resistant Staphylococcus aureus biofilms”, Lancet. 357:40-41, 2001). A biofilm inhibitor, co-administered with an antibiotic that alone is unable to clear a biofilm, for example, can disrupt the biofilm protection sufficiently to allow the antibiotic to reach the bacteria, eliminate them and prevent a recurring infection.

Biofilm inhibitors can also be employed for the treatment of acne. Acne vulgaris is the most common cutaneous disorder. Propionibacterium acnes, which is the predominant microorganism occurring in acne, reside in biofilms. Its existence in a biofilm matrix provides a protective, physical barrier that limits the effectiveness of antimicrobial concentrations (Burkhart, C. N. et. al., “Microbiology's principle of biofilms as a major factor in the pathogenesis of acne vulgaris”, International J. of Dermatology. 42:925-927, 2003). Biofilm inhibitors may be used to prevent, control, reduce, or eradicate P. acnes biofilms.

Furthermore, biofilm inhibitors can be employed to improve contact lens hygiene. Investigations on the occurrence of bacterial biofilms on contact lens have shown that up to 80 percent of contact lens wearers experienced contamination of lens cases. Bacteria protected by the biofilm can be resistant to current lens disinfectant systems (McLaughlin, B. L. et. al., “Bacterial Biofilm on contact lenses and in lens storage cases in wearers with microbial keratitis”, J. of Applied Microbiology 84(5):827-838, 1998). The use of biofilm inhibitors may prevent, control, reduce, or eradicate bacterial biofilms on contact lens.

Accordingly, for the reasons discussed above and others, there continues to be a need for a means to control biofilm and its formation.

### SUMMARY

Accordingly, the present invention provides a method for preventing, reducing, inhibiting or removing a biofilm comprising contacting the biofilm or contacting a cell capable of forming a biofilm with an effective amount of palmitoleic acid, wherein the composition prevents, reduces, inhibits or removes the biofilm.

Still, the present invention provides a method for controlling acne, comprising administering to a subject afflicted with acne an effective amount of an anti-acne composition comprising palmitoleic acid, wherein the amount of the palmitoleic acid in the anti-acne composition is sufficient to prevent, reduce, inhibit or remove a biofilm.

The present invention also provides a method for controlling a chronic bacterial infection, comprising administering to a subject in need thereof an effective amount of a composition comprising palmitoleic acid, wherein the amount is effective to prevent, inhibit, reduce, or remove a biofilm.

In addition, the present invention provides a method for identifying an agent capable of preventing, reducing, inhibiting or removing a biofilm, comprising a) contacting a biofilm with the agent, wherein the agent is a derivative of a palmitoleic acid; b) quantifying the biofilm before and after contacting the biofilm with the agent; and c) selecting the agent that prevents, reduces, inhibits or removes at least about 50% of the biofilm, wherein the agent does not inhibit bacterial growth when the concentration amount of the agent is at least twice the amount needed to prevent, reduce, inhibit, or remove at least 50% of the biofilm.

Furthermore, the present invention provides a pharmaceutical composition for reducing, preventing, inhibiting or removing biofilm formation comprising a pharmaceutically acceptable carrier or diluent and a therapeutically effective amount of 0.01 to 50% of palmitoleic acid.

The present invention also provides an animal food composition comprising palmitoleic acid, wherein the animal food composition promotes animal dental hygiene by preventing, reducing, inhibiting or removing a biofilm.

In addition, the present invention provides a dentifrice composition comprising palmitoleic acid, wherein the dentifrice composition promotes dental hygiene by preventing, reducing, inhibiting or removing a biofilm.

The present invention further provides a nebulizer for administration of a composition to a subject, wherein the composition comprises palmitoleic acid and wherein the composition prevents, reduces, inhibits or removes a biofilm.

### BRIEF DESCRIPTION OF THE FIGURES

FIG. 1 shows the chemical structure of the compound of the present invention and its analogs or derivatives.

FIG. 2 shows the HPLC/UV purification chromatograms for Hit 39 from Penstemon centranthifolius.

FIG. 3 shows the proton NMR spectra for Hit 39 from Penstemon centranthifolius.

FIG. 4 shows the HPLC/UV purification chromatograms for Hit 37 from Sambucus mexicana.

FIG. 5 shows the proton NMR spectra for Hit 37 from Sambucus mexicana.

### DETAILED DESCRIPTION

Accordingly, the present invention provides a method for preventing, reducing, inhibiting or removing a biofilm comprising contacting the biofilm or contacting a cell capable of forming a biofilm with an effective amount of palmitoleic acid, wherein the composition prevents, reduces, inhibits or removes the biofilm.

In an embodiment, the cell is selected from the group consisting of Streptococcus spp. including, but not limited to, Streptococcus mutans, Streptococcus sobrinus, Streptococcus pyogenes, and Streptococcus pneumoniae, and Escherichia coli, Pseudomonas aeruginosa, Staphylococcus aureus, Staphylococcus epidermidis, Propionibacterium acnes, Enterococcus faecium, Enterococcus faecalis, and Haemophilus influenzae.

Palmitoleic acid does not appear to be toxic as it can be readily found in foods. Palmitoleic acid can be found in, for example, animal fat products, vegetable oil, olive oil, macadamia nut oil, sea buckthorn oil and peanut butter products.

TABLE 2
Examples of palmitoleic acid in plant oils and food.

| | % Palmitoleic Acid |
|---|---|
| Animal fat, bacon grease | 2.7 |
| Olive oil | 0.3-3.5 |
| Vegetable oil, soybean lecithin | 0.4 |
| Peanut Butter | 0-8 |
| Sea buckthorn seed oil | 9.0 |
| Macadamia nut oil | 12-22 |

Palmitoleic acid can also be found in macadamia nuts. Macadamia nuts consist of approximately 75% fat, of which palmitoleic acid is approximately 12% to 22%. A reasonable serving of macadamia nuts would be approximately 50 grams. Therefore, at a minimum, an individual would consume approximately 4.5 grams of palmitoleic acid while eating a reasonable portion of macadamia nuts. An enriched diet containing macadamia nuts significantly decreases the plasma total cholesterol, low density lipoprotein cholesterol, and triacylglycerol concentrations in plasma (Yang, Baoru et. al., “Effects of dietary supplementation with sea buckthorn seed and pulp oils on atopic dermatitis”, J. Nutr. Biochem. 10:622-630, 1999).

Palmitoleic acid can also be found in sea buckthorn oil. A topical application of sea buckthorn oil on burned, scaled, wounded, and radioactively damaged skins of both humans and experimental animals have shown healing and anti-inflammatory effects (Yang, Baoru et al., “Effects of dietary supplementation with sea buckthorn seed and pulp oils on atopic dermatitis”, J. Nutr. Biochem. 10:622-630, 1999). Further, treatment with sea buckthorn reduces the serum levels of laminin, hyaluronic acid, total bile acid, collagen types III and IV in patients with liver cirrhosis (Gao, Ze-Li et al., “Effect of Sea buckthorn on liver fibrosis: A clinical study”, World J Gastroenterol. 9(7):1615-1617, 2003).

As can be appreciated, palmitoleic acid has many beneficial medicinal values. Its prevalence in food suggests that it is safe and non-toxic to animals at high doses. The disclosure herein describes a novel use for palmitoleic acid in the control of biofilm and/or its formation. Palmitoleic acid may potentially provide a safe means to control, remedy or treat a variety of conditions involving biofilms.

Plaque biofilms contribute to cavities and and periodontitis. Plaque biofilm accumulates because of bacterial colonization by Streptococci spp. such as S. mutans, S. sobrinas, S. gordonii, and Porphyromonas gingivalis, and Actinomyces spp (Demuth, D. et al. Discrete Protein Determinant Directs the Species-Species Adherence of Porphyromonas gingivalis to Oral Streptococci, Infection and Immunity, 2001, 69(9) p5736-5741; Xie, H., et al. Intergeneric Communication in Dental Plaque Biofilms. J. Bacteriol. 2000, 182(24), p 7067-7069). The primary colonizing bacteria of plaque accumulation are Streptococci spp. and P. gingivalis is a leading cause of periodontitis (Demuth, D. et al. Discrete Protein Determinant Directs the Species-Species Adherence of Porphyromonas gingivalis to Oral Streptococci, Infection and Immunity, 2001, 69(9) p5736-5741). Biofilm inhibitors can be employed to prevent microorganisms from adhering to surfaces like teeth that may be porous, soft, hard, semi-soft, semi-hard, regenerating, or non-regenerating. These surfaces can be the polyurethane material of central venous catheters, or metal, alloy, or polymeric surfaces of medical devices, or regenerating proteins of cellular membranes of mammals, or the enamel of teeth. These inhibitors can be coated on or impregnated into these surfaces prior to use, or administered at a concentration surrounding these surfaces to control, reduce, or eradicate the microorganisms adhering to these surfaces.

Therefore, another embodiment of the present invention is a method for preventing, reducing, inhibiting or removing a biofilm comprising contacting the biofilm or contacting a cell capable of forming a biofilm with an effective amount of a composition comprising palmitoleic acid, wherein the composition further comprises a dentifrice.

The present invention also provides a dentifrice composition comprising palmitoleic acid, wherein the dentifrice composition promotes dental hygiene by preventing, reducing, inhibiting or removing a biofilm. In one embodiment, the dentifrice composition is selected from the group consisting of toothpaste, toothpowder, liquid dentifrice, mouth detergent, mouthwash, troches, chewing gum, dental or gingival massage cream, dental strip, dental gel, and gargle tablet. Preferably, the dentifrice composition comprises about 0.01% to about 20% of palmitoleic acid. More preferably, the dentifrice composition comprises about 2% of palmitoleic acid.

In another embodiment, the dentifrice composition further comprises at least one antimicrobial agent. Preferably, the antimicrobial agent is an antibiotic. The antibiotic may be selected from the following group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides.

In still another embodiment, the dentifrice composition is selected from the group consisting of toothpastes, toothpowders, liquid dentifrices, mouth detergents, mouthwashes, troches, chewing gums, dental or gingival massage creams, dental strips, dental gels, and gargle tablets. In many instances, an antimicrobial or the agent controlling the biofilm alone does not resolve the medical condition or disease. The disclosure herein describes the discovery that, when used in combination with at least one antimicrobial agent, palmitoleic acid provides a significant synergistic effect.

Acne vulgaris is the most common cutaneous disorder. Propionibacterium acnes, which is the predominant microorganism present in acne, reside in biofilms. The biofilm matrix provides a protective, physical barrier that limits the effectiveness of antimicrobial against P. acnes.

The present invention, therefore, provides a method for controlling acne, comprising administering to a subject afflicted with acne an effective amount of an anti-acne composition comprising palmitoleic acid, wherein the amount of the palmitoleic acid in the anti-acne composition is sufficient to prevent, reduce, inhibit or remove a biofilm.

As used in herein, “control” or “controlling” refers to the treatment and prevention of acne. Preferably, palmitoleic acid prevents, reduces, inhibits or removes a biofilm of Propionibacterium acnes. Preferably, the antimicrobial agent is an antibiotic. The antibiotic may be selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides.

Preferably, the anti-acne composition comprises between about 0.01% to about 50% of palmitoleic acid.

The anti-acne composition may be topically administered. Preferably, the anti-acne composition is applied to the skin of the subject. The anti-acne composition may be a cream, gel, emollient or soap. Preferably, the subject is a mammal. More preferably, the mammal is a mouse or rat. Even more preferably, the mammal is a human.

In another embodiment, the concentration of palmitoleic acid in the anti-acne composition is about 0.01%, more preferably about 0.1% to about 5.0%. Because palmitoleic acid can be readily found in food and poses limited toxicity, the present invention contemplates that about 1.0% to 50% of palmitoleic acid in the anti-acne composition may be administered to control acne.

In yet another embodiment, the anti-acne composition further comprises at least one antimicrobial agent. Preferably, the antimicrobial agent is an antibiotic. The antibiotic may be selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides

Akamatsu et al., Arch Dermatol Res (1990) 282:449-454 described that acne could be treated with anti-inflammatory compounds, such as free fatty acids and metronidazole. The article taught that while a combination of palmitoleic acid and metranidazole reduces inflammatory mediators of neutrophils, neither compound alone could effectively inhibit the growth of Propionibacterium acnes. The authors used approximately 2 ug/ml of palmitoleic acid with 100 ug/ml of metranidazole to inhibit the in vitro growth of Propionibacterium acnes. The authors applied methods similar to that set forth by the National Committee for Clinical Laboratory Standards (NCCLS). Only a very small amount of palmitoleic acid was used because the authors believed that while metranidazole affected the grow of P. acnes, palmitoleic acid merely reduced the inflammation associated with acne. The present inventors determined that 2 ug/ml of palmitoleic acid would be ineffective in controlling biofilm, especially for in vivo use. This may explain why the application of an antibiotic alone to the skin of a subject with acne (even assuming that the skin naturally has palmitoleic acid), does not resolve the acne.

Willie et al., Skin Pharmacol. Appl. Skin Physiol. (2003) 16:176-187 offer another perspective on the role of palmitoleic acid. Willie et al. taught that palmitoleic acid could inhibit the growth of Gram-positive bacteria (typical minimal inhibitory concentration (MIC) value of 10-20 ug/ml), but could not inhibit the growth of Gram-negative bacteria such as Pseudomonas aeruginosa, and Escherichia coli, or Propionibacterium acnes and several methicillin-resistant strains of Staphylococcus aureus. For example, palmitoleic acid failed to inhibit the growth of P. acnes even at a high concentration, such as 125 ug/ml, using methods similar to that prescribed by the NCCLS. Willie et al. described that palmitoleic acid could inhibit the growth of gram-positive bacteria, such as strains of Staphylococcus aureus and Streptococcus salivarius, but concluded that it had no inhibitory effect on clinical strains of Staphylococcus aureus at 100 ug/ml, suggesting that palmitoleic acid might not be useful in a medical treatment. Interestingly, no synergistic effect was observed when palmitoleic acid and an antimicrobial agent, mupirocin, was used against Gram-positive bacteria.

The present invention also provides a method for controlling a chronic bacterial infection, comprising administering to a subject in need thereof an effective amount of a composition comprising palmitoleic acid, wherein the amount is effective to prevent, inhibit, reduce, or remove a biofilm.

Chronic wound infections are problematic because they are difficult to eradicate or routinely recur. Diabetic foot ulcers, venous leg ulcers, arterial leg ulcers, and pressure ulcers are the most common types of chronic wounds. Of these four types of chronic wounds, diabetic foot ulcers appear to be the most prevalent. These wounds are typically colonized by multiple species of bacteria including Staphylococcus spp., Streptococcus spp., Pseudomonas spp. and Gram-negative bacilli (Lipsky, B. Medical Treatment of Diabetic Foot Infections. Clin. Infect. Dis. 2004, 39, p.S 104-14). Even though clinical evidence has shown for quite some time that these chronic wound infections are polymicrobial, which usually indicates the presence of biofilms, it has been only recently that biofilms have been implicated as the reason these infections are very difficult to treat and eradicate (Harrison-Balestra, C. et al. A Wound-isolated Pseudomonas aeruginosa Grow a Biofilm In Vitro Within 10 Hours and Is Visualized by Light Microscopy. Dermatol Surg 2003, 29, p. 631-635; Edwards, R., et al. Bacteria and wound healing. Curr Opin Infect Dis, 2004, 17, p. 91-96). In fact, it is estimated that approximately 140,000 amputations occur each year in the United States due to chronic wound infections that cannot be successfully treated with conventional antibiotics. This is another clear unmet medical condition that conventional antibiotics have been unable to resolve. It has also been suggested that treating these infections with high doses of antibiotics over long time periods has been a contributor to the development of antibiotic resistance (Howell-Jones, R. S., et al. A review of the microbiology, antibiotic usage and resistance in chronic skin wounds. J. Antimicrob. Ther. January 2005). Biofilm inhibitors in combination therapy with antibiotics have great potential to make an impact at treating chronic wounds.

With biofilms, microbes can resist antibiotics at concentrations ranging from 1 to 1.5 thousand times higher than conventional antibiotic therapy. Similarly, during an infection, bacteria surrounded by biofilms are rarely resolved by the immune defense mechanisms. Costerton, Stewart, and Greenberg, leaders in the field of biofilms, have proposed that in a chronic infection, a biofilm gives bacteria a selective advantage by reducing the penetration of an antibiotic into the depths needed to completely eradicate the bacteria's existence. Clearly, biofilms play a significant role in chronic infections, the treatment of which represents a large market whose needs have not been met.

A typical chronic lung infection is in the lungs of cystic fibrosis patients. The principal organism found in the lungs of cystic fibrosis patients is Pseudomonas aeroginosa. The bacteria exist within a biofilm. Presently, current antibiotics, specifically antibacterial compounds, cannot provide effective treatment of chronic infections, because antibiotic therapy fails to eradicate the biofilm. The Cystic Fibrosis Foundation estimated that approximately 30,000 patients have cystic fibrosis in the United States. Research supports the conclusion that therapeutics that interfere with the formation of biofilms might assist in the treatment of these patients.

Biofilm inhibitors can be used to prevent the attachment of Helicobactor pylori to gastric epithelial cells preventing their eventual invasion into these cells or inhibiting or reducing subsequent virulence factors which result in inflammation. Biofilm inhibitors can also be used to prevent or reduce the risks associated with the virulence factors of H. pylori, for example, by reducing arterial damage which can lead to an increased risk of stroke. As treatments for urinary tract infections, biofilm inhibitors can be used to prevent, control, reduce, or eradicate the population of E. coli that reside intracellularly in bladder cells.

Examples 1, 2, 4 and 8 herein demonstrates that palmitoleic acid acts as a biofilm inhibitor by reducing the attachment of Pseudomonas aeruginosa and Staphylococcos epidermidis, Escherichia coli, Streptococcus mutans, and Streptococcus sobrinas to surfaces. The invention demonstrates that palmitoleic acid exhibits biofilm inhibition and reduction against bacteria that are genetically diverse from each other as listed in the examples. These bacteria are Gram-positive and Gram-negative and from clinical and laboratory strains. Due to the diversity of the bacteria shown in the examples of the specification, palmitoleic acid exhibits broad spectrum biofilm inhibition. Therefore, the examples are not meant to limit the scope of the invention against only the bacteria examined, but only to provide sufficient evidence that palmitoleic acid exhibits broad spectrum biofilm inhibition and reduction. The examples also specifically demonstrate that palmitoleic acid can reduce a mature biofilm with and without antibiotic. Examples 5 and 6 shows that palmitoleic acid can be applied to reduce the sustainability of pre-formed biofilms with an antibiotic. Because biofilm contributes to many chronic bacterial infections, these examples strongly support the use of palmitoleic acid and possibly its analogs or derivatives described herein in the treatment of chronic bacterial infections, such as lung and ear infections. The results of the examples and descriptions in the specification also teach the distinct difference between the methods used to discover biofilm inhibitors like palmitoleic acid and the NCCLS methods used to discover conventional antibiotics. Furthermore, it becomes clear to those skilled in the art why chronic infections are not resolved by conventional antibiotics: conventional antibiotics were identified according to the NCCLS methods which do not represent the biofilms found in chronic infections.

As demonstrated in Example 5, palmitoleic acid may be administered alone to a subject for a treatment period. After this treatment period, an antibiotic with the compound could then be administered to the subject for a combined treatment period. Alternatively, the compound could be repeatedly administered alone to the subject to continually reduce the formation of new biofilms.

In an embodiment, the composition to control the chronic bacterial infection further comprises at least one antimicrobial agent. Preferably, the antimicrobial agent is an antibiotic. The antibiotic may be selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides. Preferably, the subject is a mammal. More preferably, the mammal is a mouse or rat. Even more preferably, the mammal is a human.

Biofilms provide a structural matrix that facilitates bacterial adhesion to the inert surfaces of medical device implants and venous catheters. The bacteria also attach to mammalian cells. For the 5 million central venous catheters that are implanted each year, microscopic studies confirm that all of these catheters are coated by bacteria embedded in biofilms. More than 1 million patients develop urinary tract infections from catheters.

Accordingly, in an embodiment of the present invention, the chronic bacterial infection is selected from the group consisting of urinary tract infection, gastritis, lung infection, cystitis, pyelonephritis, arterial damage, otitis media, leprosy, tuberculosis, benign prostatic hyperplasia, chronic prostatitis, osteomyelitis, bloodstream infection, skin infection, rosacea, open wound infection, chronic wound infection, and sinus infection. The chronic bacterial infection may result from an infection of bacteria selected from the group consisting of Streptococcus spp. including but not limited to Streptococcus pyogenes, Streptococcus pneumoniae, Escherichia coli, Pseudomonas aeruginosa, Staphylococcus aureus, Staphylococcus epidermidis, Propionibacterium acnes, and Haemophilus influenzae.

The present invention further provides a nebulizer for administration of a composition to a subject, wherein the composition comprises palmitoleic acid and wherein the composition prevents, reduces, inhibits or removes a biofilm. Preferably, the composition prevents, reduces, inhibits or removes a biofilm in the lungs of the subject. Still preferably, the composition comprises about 0.1% to about 2.0% palmitoleic acid.

In addition, the present invention provides a method for identifying an agent capable of preventing, reducing, inhibiting or removing a biofilm, comprising a) contacting a biofilm with the agent, wherein the agent is a derivative of a palmitoleic acid; b) quantifying the biofilm before and after contacting the biofilm with the agent; and c) selecting the agent that prevents, reduces, inhibits or removes at least about 50% of the biofilm, wherein the agent does not inhibit bacterial growth when the concentration amount of the agent is at least twice the amount needed to prevent, reduce, inhibit, or remove at least 50% of the biofilm. Preferably, the biofilm is quantified by staining the biofilm with a dye and measuring the stained biofilm using a spectrophotometer to obtain the quantitative value for the mass of each biofilm. The biofilm is preferably a mature biofilm. That is, “mature” refers to a biofilm that is fully developed and shows resistance to known antibiotics.

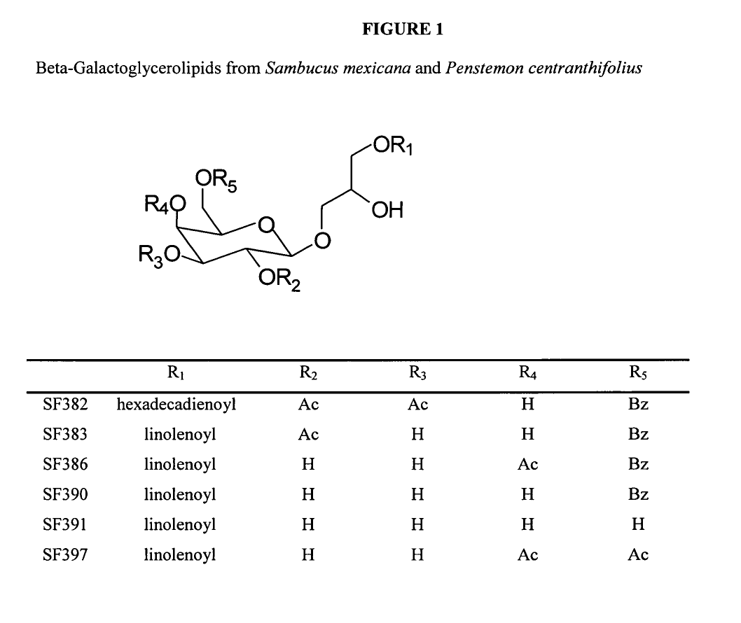

The present application also describes novel compounds that affect biofilm formation. (See FIG. 1). The compounds can prevent or reduce the formation of biofilms or reduce the sustainability of a biofilm. The methods described in the specification and the examples involve first, identifying compounds that inhibit the attachment of bacteria to surfaces (plastic or epithelial cells), which involve biofilms, and second, testing the compounds against a preformed biofilm that is resistant to an antibiotic. This procedure simulates chronic mammalian infections, by exposing the bacteria to the biofilm inhibitor and determining if the inhibitor can reduce or modify the biofilm with or without the presence of an antibiotic.

Other analogs and derivatives of palmitoleic could potentially be used to control biofilm. Accordingly, the present inventor has produced a compound library using the method previously described (Eldridge, G. R. et. al., 2002; Cremen, Peadar A. et. al., 2002). The compound samples have corresponding preparative HPLC elution conditions that are tracked with a bar code system, so that the exact gradient elution conditions of any sample in the library could be recalled. The ability to rapidly isolate the compounds in the active samples is achieved by utilizing starting conditions from the preparative HPLC separation and applying these to the semi-preparative HPLC system. With this information, precise starting HPLC conditions are used to lower the gradient elution conditions to rapidly develop a method to isolate the compounds in the active samples. The individual compounds are then submitted for re-screening in the biofilm inhibition assays to identify the active compound(s). By “biofilm inhibition”, it is meant that the compound prevents, reduces, inhibits or removes a biofilm formation or a biofilm that is fully developed.

After screening 12,000 samples produced from a compound library of plants according to similar methods as described in Example 1, samples that exhibited biofilm inhibition were identified. As is evident from the Biofilm Inhibiton Assay results presented in Table 1, one compound from Hit 39, SF382 confirmed biofilm inhibition after purification. As can be seen from the adjacent column (bacterial growth prior to rinsing the assay wells), this inhibition is not due to the inhibition of bacterial growth. Thus, the percentage of bacteria attached (within the biofilm) versus free-floating compared to untreated (negative) control has shifted toward free-floating upon addition of the treatment. Compound SF382 was particularly active, reducing the formation of biofilm by 50% compared to negative control. Compound SF382 is represented by structure (I).

As can be seen in the second part of Table 1, the Detachment Assay results, the selected compounds detached significant portions of a preformed biofilm when compared to the untreated (negative) control. Compound SF397 was particularly active with a detachment of 27% after one hour incubation time.

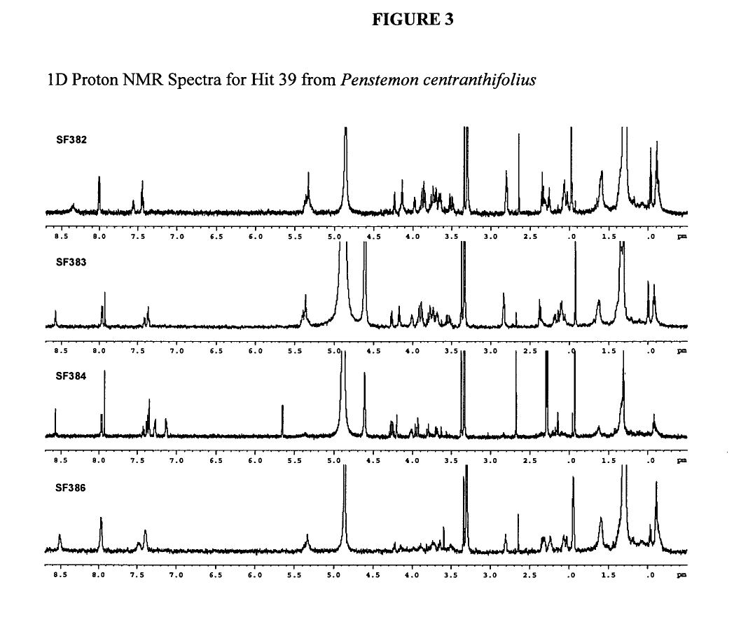

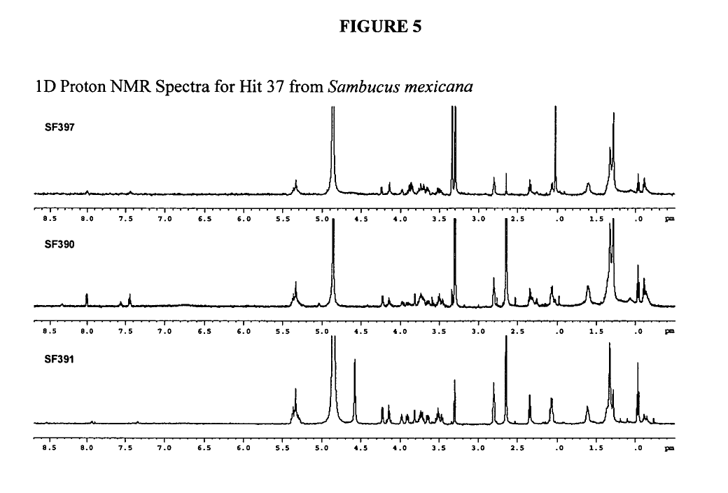

FIGS. 3 and 5 show the analysis of an NMR data; the data reveal that the active compounds isolated from Hits 37 and 39 are structurally similar. They all consist of a beta-galactoglyceryl moiety esterified via the terminal glycerol hydroxy functionality to a fatty acid. Aromatic functionality has not been previously reported. An extensive literature search has revealed that the benzoyl containing compounds SF382, SF383, SF386, SF390, and SF397 are novel structures. While compound SF391 is known in the art, the present inventors have shown herein that this compound can prevent, reduce, inhibit or remove a biofilm. Similarly, none of structures described herein have previously been reported as biofilm inhibitors.

Compound SF382 exhibited potent biofilm inhibition. SF382 contains a hexadecadienoyl fatty acid side chain. The other compounds carry a linolenoyl fatty acid side chain. The hexadecadienoyl appears to be a significant contributor to biofilm inhibition. This hypothesis lead to the discovery of palmitoleic acid.

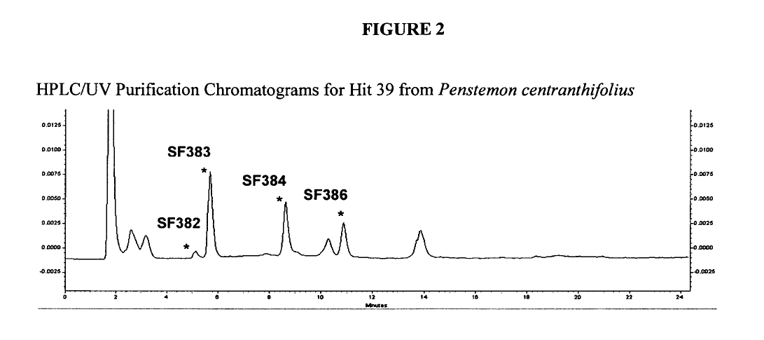

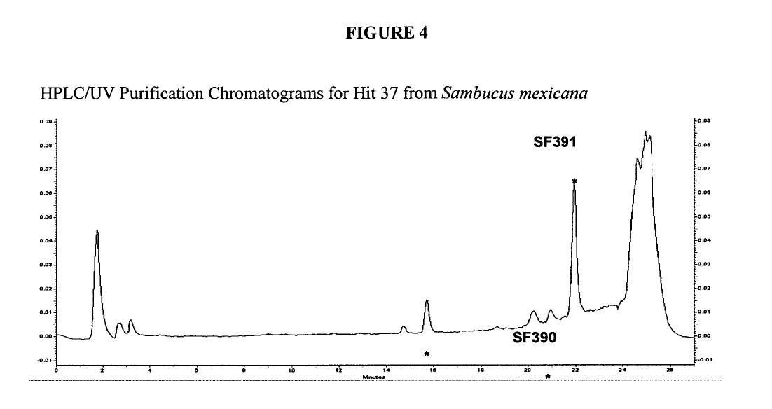

FIGS. 2 and 4 illustrate the HPLC collection chromatograms of the compounds isolated from two samples, Hits 39 and 37. The active compounds from Hit 39 were isolated from Penstemon centranthifolius (Scorphulariales) collected in North America. The active compounds from Hit 37 were isolated from Sambucus mexicana (Caprifoliaceae) collected in North America. The chemical structures of the biofilm inhibitors are determined using a 600 MHz NMR spectrometer equipped with a CapNMR probe.

Hit 37 and 39 were generated from Sambucus mexicana Presl. (leaf and flower extract) and from Penstemon centranthifolius Benth. (whole plant extract), respectively. The common names for the source plants are Blue Elderberry and Scarlet bugler (or Beardtongue), respectively. Sambucus is in the Family Caprifoliaceae, Order Dipsacales; Penstemon is from the family Scrophulariaceae, Order Scrophulariales. Both orders are in the Class Asteridae. The Asteridae also contains Asters, Dogbanes, Mints and other taxa known to produce a wide range of chemical repellants. Because Classes and Orders are fairly large taxonomic groupings it is safe to suggest that while the two plants are clearly related, the relationship is somewhat distant.

In addition to being from the same Class, the two plants share similar biogeography. Both are woodland plants from California and both tend to grow in open spaces below 3000 meters. The Elderberry tends towards moister climates than the Beardtongue, but neither is found in a truly arid environment. It would not be surprising to find that two plants in the same taxonomic Class and sharing the same environment would evolve similar compounds.

TABLE 1
Biofilm Inhibition and Detachment of the Isolated
Compounds from the Active Samples

| | | Biofilm Inhibition | | Detachment Assay | | |
|---|---|---|---|---|---|---|
| | | Assay | | % Growth | % Growth | |
| Active | Isolated | % | % Biofilm | Before | After | % |
| Sample | Compound | Growth | Inhibition | Compound | Compound | Detachment |
| Hit 39 | SF382 | 7 | 50 | 4 | 14 | 15 |
| | SF383 | −4 | 4 | 1 | 1 | 15 |
| | SF384 | 2 | 19 | 5 | 11 | 19 |
| Hit 37 | SF390 | −4 | 21 | 0 | 4 | 15 |
| | SF391 | −7 | −1 | −4 | 3 | 11 |
| | SF397 | 0 | 5 | 3 | 9 | 27 |
| Negative | | 100 | 0 | 100 | 100 | 0 |
| Control | | | | | | |

Analogs and moieties of palmitoleic acid were tested as mentioned in Example 1. The analogs also include oleic acid, ricinoleic acid, 1-O-hexyadecyl-2,3-dipalmitoyl-rac-glycerol, and 1-palmitoyl-2-oleyl-3-linoleoyl-rac-glycerol, which each exhibited biofilm inhibition from approximately 20% to 40%. Preferably, the analog has a 16-carbon conjugated chain. Unlike palmitoleic acid and compound SF382, palmitic acid lacks the double bond conjugation and does not inhibit the formation of biofilms.

FIG. 1 lists the compounds prepared and tested for biofilm inhibition as described in Example 1. The compounds were prepared based on the discovery of the novel function of palmitoleic acid in controlling biofilm as described herein. The compounds are represented by the following structures:

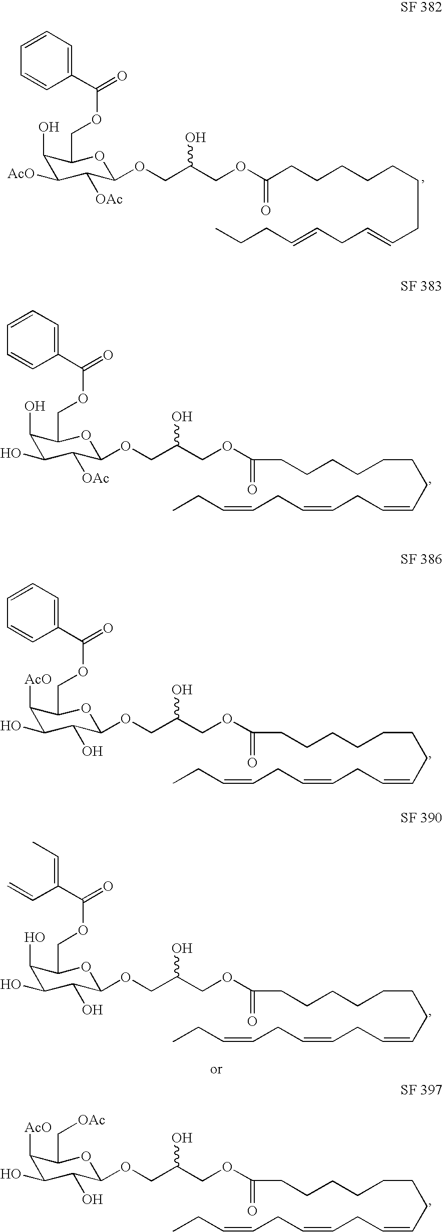

A composition comprising one or more of these compounds may be used to affect biofilm formation. The composition may further comprise at least one antimicrobial agent. Preferably, the antimicrobial agent is an antibiotic. The antibiotic may be selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides.

In another embodiment, the composition further comprises a pharmaceutically acceptable carrier and diluent. This pharmaceutical composition may further comprise at least one antimicrobial agent. Preferably, the antimicrobial agent is an antibiotic. The antibiotic may be selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides.

Furthermore, the present invention provides an animal food composition comprising palmitoleic acid, wherein the animal food composition promotes animal dental hygiene by preventing, reducing, inhibiting or removing a biofilm. The biofilm may be a dental plaque. In an embodiment, the animal food composition is a dry or semi-moist pet food composition. For example, the pet food composition is a pet biscuit. Alternatively, the pet food composition is applied to the surface of a chewable pet toy. Preferably, the pet food composition is a dog or cat food.

### EXAMPLES

The following examples illustrate the testing of palmitoleic acid and related compounds and the preparation of formulations comprising palmitoleic acid. The examples demonstrate the many uses of palmitoleic acid and are not intended to limit the scope of the present invention.

### Example 1

Effect of Palmitoleic Acid on Biofilm Inhibition with Pseudomonas aeruginosa PA01 and Staphylococcus epidermidis ATCC 29641.

In vitro biofilm inhibition was evaluated using a high-throughput method. This method uses standard pre-sterilized 96-well polystyrene microtiter plates. Each well was filled to a final volume of 200 μl. Initially, a concentrated compound solution is transferred into each well, except those which are used as controls, to achieve an appropriate testing concentration, typically 10 μg/ml. 150 μl of sterile media was then added, followed by 50 μl of bacterial inoculum. The plates were then placed on a shaker for 24 hours.

After the allotted growth time, the samples were removed from the shaker, rinsed and stained. The rinse involves first draining the wells, filling each well with PBS, and draining each well again. The rinse removes suspended cells from the system. The biofilm was then stained with crystal violet for 20 minutes. The wells were each rinsed 4 times to remove any excess stain from the system and then eluted with 250 μl of ethanol. The elution step improves the detection of the stain during the analyses. The plate was then immediately analyzed with a microtiter plate reader. The samples and controls were analyzed in triplicate. Each plate contains negative and positive controls. The test samples were compared to the positive and negative controls to detect any reduction in the total amount of biofilm.

Palmitoleic acid at 10 μg/mL resulted in 55% biofilm inhibition for P. aeruginosa PA01 and 80% biofilm inhibition for S. epidermidis ATCC 29641.

### Example 2

Palmitoleic acid and Biofilm Formation with Wild-Type Escherichia coli JM109.

Biofilm inhibition experiments were conducted using an assay adapted from the reported protocol described in Pratt and Kolter, 1998, Molecular Microbiology, 30: 285-293; Li et al., 2001, J. Bacteriol., 183: 897-908. E. coli JM109 was grown in LB with or without 0.2% glucose in 96 well plates at 37° C. for two days without shaking. To quantify the biofilm mass, the suspension culture was poured out and the biofilm was washed three times with water. The biofilm was stained with 0.1% crystal violet for 20 minutes. The plates were then washed three times with water. OD reading at 540 nm was measured to quantify the biofilm mass at the bottom of the wells. Then 95% ethanol was added to dissolve the dye at the bottom and on the wall and the OD reading at 540 nm was measured to quantify the total biofilm mass. To study the overall effect of palmitoleic acid (3.6 mg/mL in 100% ethanol as stock solution), it was added with the inoculation and a time course of biofilm mass was measured. To study if the compound can cause biofilm removal, it was added one day after inoculation. Appropriate amounts of 100% ethanol were added to each sample to eliminate the effect of solvent. Four plates were prepared for each study to give a time course. Each condition had 3-4 replicates on each plate.

Palmitoleic acid had no inhibitory effect on the growth of E. coli JM109 when compared to controls, demonstrating that palmitoleic acid is not an antibacterial compound. However, when palmitoleic acid was added with inoculation of E. coli, the compound, at approximately 20 μM, inhibited the total biofilm formation by about 60% reduction as compared to the controls.

### Example 3

Antibacterial effect of palmitoleic acid on methicillin-resistant Staphylococcus aureus (ATCC 33591 and 78-13607A).

Using the appropriate NCCLS procedures, the antibacterial effect of palmitoleic acid on two strains of methicillin-resistant Staphylococcus aureus (ATCC 33591 and 78-13607A) was studied at 32 μg/mL. For both S. aureus ATCC 33591 and S. aureus ATCC 78-13607A, palmitoleic acid had no inhibitory effect represented by an MIC (minimal inhibitory concentration) of greater than 32 μg/mL. These results along with the results described in Example 2, further supports that palmitoleic acid is not an antibacterial compound.

### Example 4

Effect of Palmitoleic Acid on Biofilm Inhibition of a clinical strain of Escherichia coli UTI89.

Dose response experiments were conducted on palmitoleic acid as described in Example 2 at 4, 8, 16, and 32 ug/mL with E. coli UT189 in LB media.

Palmitoleic acid inhibited biofilm of E. coli UT189 by approximately 10% (4 μg/mL), 55% (8 μg/mL), 70% (16 μg/mL), and 80% (32 μg/mL) compared to controls.

### Example 5

Effect of palmitoleic acid and Tobramycin on Biofilm formation with Pseudomonas aeruginosa.

Biofilm formation of P. aeruginosa was evaluated using a standardized biofilm method with a rotating disk reactor (RDR). This method provides a model resembling the formation of biofilms in cystic fibrosis patients. The rotating disk reactor consists of a one-liter glass beaker fitted with a drain spout. The bottom of the vessel contains a magnetically driven rotor with six 1.27 cm diameter coupons constructed from polystyrene. The rotor consists of a star-head magnetic stir bar upon which a disk was affixed to hold the coupons. The vessel with the stir bar was placed on a stir plate and rotated to provide fluid shear. A nutrient solution (AB Trace Medium with 0.3 mM glucose, see Table 3 below for composition) was added through a stopper in the top of the reactor at a flow rate of 5 ml/min. The reactor volume was approximately 180 ml and varied slightly between reactors depending on the placement of the drain spout and the rotational speed of the rotor. At a volume of 180 ml, the residence time of the reactors was 36 minutes. The reactors were operated at room temperature (c.a. 26° C.).

TABLE 3
Composition of the AB Trace Medium used for the RDR test.

| Component | Formula | Concentration (g/l) |
|---|---|---|
| Disodium phosphate | Na₂HPO₄ | 6.0 |
| Monopotassium phosphate | KH₂PO₄ | 3.0 |
| Sodium Chloride | NaCl | 3.0 |
| Ammonium sulfate | (NH₄)₂SO₄ | 2.0 |
| Magnesium chloride | MgCl₂ | 0.2 |
| Glucose | C₆O₁₂H₆ | 0.054 |
| Calcium chloride | CaCl₂ | 0.010 |
| Sodium sulfate | Na₂SO₄ | 0.011 |
| Ferric chloride | FeCl₃ | 0.00050 |

For each test, two RDRs were operated in parallel with one receiving palmitoleic acid and the other serving as an untreated control. The RDRs were sterilized by autoclave, then filled with sterile medium and inoculated with Pseudomonas aeruginosa strain PAO1. The reactors were then incubated at room temperature in batch mode (no medium flow) for a period of 24 hours, after which the flow was initiated for a further 24 hour incubation. Palmitoleic acid was dissolved in 10 ml ethanol to achieve a concentration of 1.8 mg/ml. After the 48 hours of biofilm development described above, the 10 ml of ethanol containing palmitoleic acid was added to the reactor to achieve a final concentration of approximately 100 μg/ml. Control reactors received 10 ml of ethanol. The reactors were then incubated for an additional 24 hours in batch (no flow) mode. After this incubation period, the six coupons were removed from each reactor and placed in 12-well polystyrene tissue culture plates with wells containing either 2 ml of a 100 μg/ml tobramycin solution or 2 ml of phosphate-buffered saline (PBS). These plates were incubated at room temperature for two hours. The coupons were then rinsed by three transfers to plates containing 2 ml of fresh PBS. For each two RDR reactors run in parallel, four sets of three coupons were obtained: one set with no test compound treatment and no tobramycin treatment, one set with no test compound treatment and tobramycin treatment, one set treated with a test compound treatment and no tobramycin treatment, and one set treated with a test compound treatment and tobramycin. After rinsing, one coupon of each set of three was stained with a LIVE/DEAD® BacLight™ Bacterial Viability Kit (Molecular Probes, Eugene Oreg.) and imaged using epifluorescent microscopy. The remaining two coupons were placed in 10 ml of PBS and sonicated for five minutes to remove and disperse biofilm cells. The resulting bacterial suspensions were then serially diluted in PBS and plated on tryptic soy agar plates for enumeration of culturable bacteria. The plates were incubated for 24 hours at 37° C. before colony forming units (CFU) were determined.

The combined treatment of palmitoleic acid and tobramycin resulted in a 60% reduction of CFUs for P. aeruginosa compared to treatment with tobramycin alone. This clearly demonstrates that palmitoleic acid increased biofilm susceptibility to tobramycin by modifying the biofilm. As a comparison to multiple published clinical studies, these results with palmitoleic acid and tobramycin demonstrate that improved lung function (FEV or forced expiratory volume) and decreased average CFU (density) in sputum from patients with cystic fibrosis would be observed in a combination therapy involving these compounds (Ramsey, Bonnie W. et. al., “Intermittent administration of inhaled tobramycin in patients with cystic fibrosis”, New England J. Medicine 340(1):23-30, 1999; Saiman, L. “The use of macrolide antibiotics in patients with cystic fibrosis”, Curr Opin Pulm Med, 2004, 10:515:523; Pirzada, O. et al. “Improved lung function and body mass index associated with long-term use of Macrolide antibiotics.”, J. Cystic Fibrosis, 2003, 2, p. 69-71). Using the endpoints listed in these publications and used in Cystic Fibrosis clinical trials, this example demonstrates that a combined treatment of tobramycin and palmitoleic acid would provide benefit to Cystic Fibrosis patients or other people suffering from chronic lung infections. The research results of this example also demonstrate that palmitoleic acid in combination with an antibiotic would remove biofilms from teeth, skin, and other surfaces.

### Example 6

Effect of Palmitoleic Acid on Mature Biofilms of clinical strains of P. aeruginosa clinical isolates isolated from cystic fibrosis patients were passed twice on tryptic soy agar with 5% sheep blood after retrieval from −80° C. and then grown overnight in CAMHB. After dilution of a culture to 0.5 McFarland in broth medium, 100 μl was transferred in triplicate to wells of a flat-bottom 96-well microtiter plate. Bacterial biofilms were formed by immersing the pegs of a modified polystyrene microtiter lid into this biofilm growth plate, followed by incubation at 37° C. for 20 hours with no movement.

Peg lids were rinsed three times in sterile water, placed onto flat-bottom microtiter plates containing biofilm inhibitors at 10, 20, and 30 ug/ml in 100 μl of CAMHB per well and incubated for 40 to 48 h at 37° C.

Pegs were rinsed, placed in a 0.1% (wt/vol) crystal violet solution for 15 min, rinsed again, and dried for several hours. To solubilize adsorbed crystal violet, pegs were incubated in 95% ethanol (150 μl per well of a flat-bottom microtiter plate) for 15 min. The absorbance was read at 590 nm on a plate reader. The wells containing palmitoleic acid were compared to negative controls. Negative controls were prepared as stated above but without palmitoleic acid.

Palmitoleic acid caused approximately 50% to 70% detachment of the mature biofilms at 10, 20, and 30 ug/ml against three clinical strains of P. aeruginosa.

### Example 7

In Vitro Cytoxicity Assessment of palmitoleic acid in Human Hepatocytes.

Palmitoleic acid was tested to assess its cytotoxicity in human hepatocellular carcinoma cells (HepG2). Palmitoleic acid was tested at two concentrations of 30 and 60 μM in HepG2 cells in triplicate. Gross cytotoxicity was assayed by fluorometric detection in response to mitochondrial activity according to Nociari M M, et al. (1998) J. Immunol. Met. 213:157. Chlorpromazine was used as a positive control.

Cytotoxicity of HepG2 cells was not observed at palmitoleic acid concentrations of 30 or 60 μM.

### Example 8

Effect of Palmitoleic Acid on Biofilm Growth and Inhibition with Streptococcus mutans 25175 and Streptococcus sobrinus 6715.

Palmitoleic acid was tested against S. mutans 25175 and S. sobrinus 6715 at a concentration of 80 μg/ml using the method described in Example 2. The use of 1 mL polycarbonate tubes were used in place of 96 well polysterene microtiter plates.

Testing palmitoleic acid at 80 μg/mL against S. mutans 25175 and S. sobrinus 6715 showed greater than 75% biofilm growth inhibition.

### Example 9

In Vivo PK Study of Palmitoleic Acid in Rats.

Two groups of animals were prepared to evaluate palmitoleic acid. Palmitoleic acid was evaluated for two dose routes consisting of 50 mg/kg (IV) and 100 mg/kg (Oral). Three animals were assigned to the IV route group and two animals were assigned to the oral route group. Prior to dosing, a baseline blood sample was taken from each animal. At time zero, a single bolus dose of palmitoleic acid in 10% Cremophor (BASF) was given to each animal. Blood samples were taken from each animal at 1, 4, 12 and 24 hours post dosing. All blood samples were processed to plasma.

The plasma samples were extracted by adding internal standard at a concentration of 10 μg/mL to achieve a final concentration of 2 μg/mL when reconstituted. A solution of 80:20 methanol:chloroform was used to cause protein precipitation. The samples were then vortexed and centrifuged. The supernatant was decanted and the solvent was evaporated off. Samples were then analyzed and quantified by LC/MS.

The reported AUC for IV dosed rats was 123 μg/ml/hr and the AUC for the orally dosed rats was 219.71 μg/ml/hr. These data demonstrate that palmitoleic acid is in sufficient quantities in serum of rats to demonstrate biofilm inhibition or reduction.

### Example 10

Palmitoleic Acid, 2% Topical Biofilm Inhibitor Formulation.

This invention provides topical biofilm inhibitor preparations containing 2% by weight palmitoleic acid. A similar formulation could be prepared as listed in this example with an antibiotic.

Components of the oil phase are heated to 65° C., with constant stirring, until molten. Palmitoleic acid is added to the oil phase and stirred for 5 minutes. The aqueous phase is heated to 65° C. and stirred until complete solution is achieved. The aqueous phase is mixed with oil phase at the same temperature for 10 minutes. The cream is stirred and cooled to 40-45° C., before a vacuum of 1 bar is pulled. Stirring is continued until the cream reaches 25° C.

Formulation A

| Ingredients | Parts By Weight |
|---|---|
| Oil Phase | |
| Mineral Oil | 50.9 |
| Stearyl Alcohol | 3.5 |
| Cetyl Alcohol | 3.5 |
| Phenoxy Ethanol | 0.5 |
| Benzyl Alcohol | 1.0 |
| Palmitoleic Acid | 2.0 |
| Aqueous Phase | |
| Polyethylene Glycol | 6.0 |
| Xanthum Gum | 0.2 |
| Water | 32.4 |

### Example 11

Topical antibacterial gels were prepared containing 5% of palmitoleic acid by weight with and without antibiotic.

Formulation A. 0.5 gram of palmitoleic acid was dissolved in 5.5 grams of ethanol. 0.2 grams of hydroxypropyl methylcellulose was added with gentle stirring until a homogenous solution was obtained. 3.74 grams of water was then added with gentle shaking.

Formulation B. 0.5 gram of palmitoleic acid was dissolved in 5.5 grams of ethanol. 0.2 grams of azithromycin was dissolved in this solution. 0.2 grams of hydroxypropyl methylcellulose was added with gentle stirring until a homogenous solution was obtained. 3.74 grams of water was then added with gentle shaking.

Formulations A and B were stored for thirty days at 2° C. to 8° C., room temperature (approximately 22° C.), and at 30° C. Both formulations at each storage condition remained homogenous for thirty days.

### Example 12

Palmitoleic Acid, 2% Toothpaste Formulation

Toothpaste preparations were prepared containing 2% palmitoleic acid with and without antibiotic and with and without polymer. Polymer, Gantrez S-97, was added to improve retention of palmitoleic acid and antibiotic on teeth.

All of the dry ingredients were mixed together. Glycerin was slowly added while mixing. An aliquot of water was added slowly and thoroughly mixed. Peppermint extract was added and then the rest of the water was added while mixing. Palmitoleic acid and antibiotic were then added until homogenous.

Formulation A

| Ingredients | Parts By Weight |
|---|---|
| Sorbitol | 20.0 |
| Glycerin | 22.0 |
| Silica | 20 |
| Sodium lauryl sulfate | 2.0 |
| Xanthum gum | 1 |
| Palmitoleic Acid | 2.0 |
| Peppermint extract | 1.0 |
| Sodium fluoride | 0.3 |
| Water | 31.7 |

Formulation B

| Ingredients | Parts By Weight |
|---|---|
| Sorbitol | 20.0 |
| Glycerin | 22.0 |
| Silica | 20 |
| Sodium lauryl sulfate | 2.0 |
| Xanthum gum | 1 |
| Palmitoleic Acid | 2.0 |
| Peppermint extract | 1.0 |
| Sodium fluoride | 0.3 |
| Gantrez S-97 | 2.5 |
| Water | 29.2 |

Formulation C

| Ingredients | Parts By Weight |
|---|---|
| Sorbitol | 20.0 |
| Glycerin | 22.0 |
| Silica | 20 |
| Sodium lauryl sulfate | 2.0 |
| Xanthum gum | 1 |
| Palmitoleic Acid | 2.0 |
| Triclosan | 0.3 |
| Peppermint extract | 1.0 |
| Sodium fluoride | 0.3 |
| Gantrez S-97 | 2.5 |
| Water | 28.9 |

Formulations A, B, and C were prepared and stored for thirty days at 2° C. to 8° C., room temperature (approximately 22° C.), and at 30° C.

### Example 13

Palmitoleic Acid, 2% Mouth Rinse Formulation.

This invention provides an oral care mouth rinse preparation containing 2% by weight palmitoleic acid with the following formulation.

| Ingredients | Parts By Weight |
|---|---|
| Palmitoleic Acid | 2.00 |
| Ethanol | 10.00 |
| Propylene glycol | 7.00 |
| Sorbitol (70%) | 10.00 |
| Glycerol | 9.00 |
| Sodium lauryl sulfate | 0.30 |
| Tauranol | 0.20 |
| Flavor | 0.10 |
| Water | 61.4 |

### Example 14

Palmitoleic Acid, Pharmaceutical Suspension Formulation for Aerosol Administration

This invention provides a suspension aerosol formulation comprising a therapeutically effective amount of about 0.1 to 0.5% by weight of palmitoleic acid, about 5 to 12% by weight of ethanol, about a 0.1 to 0.5% by weight of oleic acid, and a propellant comprising HFC 227.

The palmitoleic acid suspension aerosol formulation of this invention can be prepared by first placing the palmitoleic acid in an aerosol vial. A mixture of the propellant, oleic acid and ethanol can then be added, and the palmitoleic acid dispersed in the mixture. This formulation could be used to assist in the reduction and prevention of bacterial biofilms in the lungs.

### Example 15

Palmitoleic Acid, Pharmaceutical Formulation for Nebulization

Solutions were prepared comprising 2 mg/ml and 10 mg/ml of palmitoleic acid in ethanol/propylene glycol/water (85:10:5). These solutions were nebulized separately by a ProNeb Ultra nebulizer manufactured by PAR1. The nebulized solutions were collected in a cold trap, processed appropriately, and detected by mass spectrometry. Palmitoleic acid was recovered from both formulations demonstrating that nebulization can be used to deliver this compound to patients with lung infections.

### Example 16

Palmitoleic Acid, 2% Liquid Soap Formulation

This invention provides a liquid soap preparation containing 2% by weight palmitoleic acid with the following formulation. This liquid soap could be used daily on the face prior to going to sleep to prevent and reduce facial acne.

| Ingredients | Parts By Weight |
|---|---|
| Palmitoleic Acid | 2.0 |
| Ethanol | 1.5 |
| Propylene glycol | 3.0 |
| Cellulose | 1.5 |
| Glycerol | 16.0 |
| Tetrasodium EDTA | 0.30 |
| Triethylamine | 10.0 |
| Water | 65.7 |

### Example 17

Palmitoleic Acid, 2% Topical Burn and Wound Healing Formulation

This invention provides a topical preparation for treating burns and healing wounds containing 2% by weight palmitoleic acid with the following formulation.

| Ingredients | Parts By Weight |
|---|---|
| Palmitoleic Acid | 2.0 |
| Vitamin E | 0.3 |
| Polyethylene glycol 400 | 49.8 |
| Polyethylene glycol 4000 | 44.9 |
| Water | 3.0 |

### Example 18

Dental Care Pet Food with Palmitoleic Acid

This invention provides a pet food composition containing palmitoleic acid.

It is preferred that the pet food of the present invention be a dry or semi-moist pet food, preferably a biscuit, but the description applies generally to dry pet foods such as pelletized dog or cat foods.

The manufacture of such pet foods, particularly pet biscuits, generally involves the step of shaping and baking a dough containing the desired ingredients. The dental care biscuits of the present invention can be made from any suitable dough according to conventional techniques known in the art.

The dough compositions may comprise, in addition to palmitoleic acid, at least one flour, meal, fat and water. A conventional dough for dog biscuits may also comprise particles of meat and/or meat byproducts and fat solids. Alternatively, the palmitoleic acid can be applied to the surface of the biscuit after baking, as a 1-50% solution in water.

The dough ingredients are admixed at a temperature of about 15° C. to about 30° C. The dough pieces can be baked using any suitable or conventional equipment and conditions in the range of about 100° C. to about 300° C. with subsequent drying to produce the desired moisture content in the final product.

### REFERENCES

Costerton, J. W. et. al. 1999. Bacterial Biofilms: A Common Cause of Persistent Infections. Science. 284:1318-1322.
Centers for Disease Control and Prevention 2002. Guidelines for the Prevention of Intravascular Catheter-Related Infections. MMWR. 51:RR-10.
Maki, D. G. et. al. 1997. Prevention of Central Venous Catheter-Related Bloodstream Infection by Use of an Antiseptic-Impregnated Catheter. Ann. Int. Med. 127(4):257-266.
Darouiche, R. et. al. 1999. A Comparison of Two Antimicrobial-Impregnated Central Venous Catheters. New Engl. Jour. Med. 340(1):1-8.
Raad, I, et. al. 1997. Central Venous Catheters Coated with Minocycline and Rifampin for the Prevention of Catheter-Related Colonization and Bloodstream Infections. Ann. Int. Med. 127:267-274.
Hanna, H. A., et. al. 2003. Antibiotic-Impregnated Catheters Associated with Significant Decrease in Nosocomial and Multidrug-Resistant Bacteremias in Critically Ill Patients. Chest. 124(3):1030-1038.
Pendland, S. L. et. al. 2002. In vitro synergy testing of levofloxacin, ofloxacin, and ciprofloxacin in combination with aztreonam, ceftazidime, or piperacillin against Pseudomonas aeruginosa. Diag. Micro. Inf Dis. 42:75-78.
Jones, S. M., et. al. 2001. Effect of vancomycin and rifampicin on methicillin-resistant Staphylococcus aureus biofilms. Lancet. 357:40-41.
CDC. Update: Investigation of Bioterrorism-Related Anthrax and Interim Guidelines for Exposure Management and Antimicrobial Therapy. October 2001. MMWR 2001. 50:909-919.
Price, L. B., et. al. 2003 In vitro selection and characterization of Bacillus anthracis mutants with high-level resistance to ciprofloxacin. Antimicrob. Agents Chemother. 57:2362-2365.
Ramsey, Bonnie W. et. al. 1999. Intermittent Administration of Inhaled Tobramycin in Patients with Cystic Fibrosis. New England J. Medicine. 340(1):23-30.
Ramage, Gordon et. al. 2003. Formation of Propionibacterium acnes biofilms on orthopaedic biomaterials and their susceptibility to antimicrobials. Biomaterials. 24:3221-3227.
Burkhart, Craig N. et. al. 2003. Microbiology's principle of biofilms as a major factor in the pathogenesis of acne vulgaris. International J. of Dermatology. 42:925-927.
McLaughlin, Borlace L. et. al. 1998. Bacterial Biofilm on contact lenses and in lens storage cases in wearers with microbial keratitis. J. of Applied Microbiology 84(5):827-838.
Landa, H. C. et. al. 1998. Efficacy of ophthalmic solutions to detach adhering Pseudomonas aeruginosa from contact lenses. 17(3):293-300.
Akamatsu, H. et. al. 1990. The inhibition of free radical generation by human neutrophils through the synergistic effects of metronidazole with palmitoleic acid: a possible mechanism of action of metronidazole in rosacea and acne. Archives of Dermatological Research 282:449-454.
Wille, J. J. et. al. 2003. Palmitoleic Acid Isomer (C16:1A6) in Human Skin Sebum Is Effective against Gram-Positive Bacteria. Skin Pharmacol Appl Skin Physiol. 16:176-187.
Yang, Baoru et. al. 1999. Effects of dietary supplementation with sea buckthorn (Hippophae rhamnoides) see and pulp oils on atopic dermatitis. J. Nutr. Bichem. 10:622-630.
Gao, Ze-Li et. al. 2003. Effect of Sea buckthorn on liver fibrosis: A clinical study. World J. Gastroenterol. 9(7):1615-1617
Eldridge, G. R. et. al. 2002. High-throughput method for the production and analysis of large natural product libraries for drug discovery. Analytical Chemistry. 74(16):3963-3971.
Cremen, Peadar A. et. al. 2002. High-Throughput Analysis of Natural Product Compound Libraries by Parallel LC-MS Evaporative Light Scattering Detection. Analytical Chemistry. 74(21):5492-5500.
Slack, J. M. 2000. Stem Cells in Epithelial Tissues. Science. 287:1431-1433.

What is claimed:

1. A method for preventing, reducing, inhibiting or removing a biofilm comprising contacting the biofilm or contacting a cell capable of forming a biofilm with an effective amount of a composition comprising palmitoleic acid, wherein the composition prevents, reduces, inhibits or removes the biofilm.

2. The method of claim 1, wherein the cell is selected from the group consisting of Streptococcus mutans, Streptococcus sobrinus, Streptococcus pyogenes, Streptococcus pneumoniae, Escherichia coli, Pseudomonas aeruginosa, Staphylococcus aureus, Staphylococcus epidermidis, Propionibacterium acnes, Enterococcus faecium, Enterococcus faecalis, and Haemophilus influenzae.

3. The method of claim 1, wherein the composition comprises about 0.1% to about 20% of palmitoleic acid.

4. A method for controlling acne, comprising administering to a subject afflicted with acne an effective amount of an anti-acne composition comprising palmitoleic acid, wherein the amount of the palmitoleic acid in the anti-acne composition is sufficient to prevent, reduce, inhibit or remove a biofilm.

5. The method of claim 4, wherein the composition prevents, reduces, inhibits or removes a biofilm of Propionibacterium acnes.

6. The method of claim 4, wherein the antimicrobial agent and palmitoleic acid or its derivative provide a synergistic effect.

7. The method of claim 4, wherein the antimicrobial agent is an antibiotic.

8. The method of claim 7, wherein the antibiotic may is selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides.

9. The method of claim 4, wherein the amount of palmitoleic acid is between about 0.5% to about 50%.

10. The method of claim 4, wherein the anti-acne composition is topically administered.

11. The method of claim 10, wherein the anti-acne composition is applied to the skin of the subject.

12. The method of claim 4, wherein the anti-acne composition is a cream, gel, emollient, or soap.

13. The method of claim 4, wherein the subject is a mammal.

14. The method of claim 13, wherein the mammal is a mouse or rat.

15. The method of claim 13, wherein the mammal is a human.

16. The method of claim 4, wherein the anti-acne composition further comprises at least one antimicrobial agent.

17. The method of claim 16, wherein the anti-acne composition prevents, reduces, inhibits or removes a biofilm of Propionibacterium acnes.

18. The method of claim 16, wherein the antimicrobial agent and palmitoleic acid or its derivative provide a synergistic effect.

19. The method of claim 16, wherein the antimicrobial agent is an antibiotic.

20. The method of claim 19, wherein the antibiotic may is selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides.

21. The method of claim 16, wherein the amount of palmitoleic acid is between about 0.5% to about 50%.

22. The method of claim 16, wherein the anti-acne composition is topically administered.

23. The method of claim 22, wherein the anti-acne composition is applied to the skin of the subject.

24. The method of claim 16, wherein the anti-acne composition is a cream, gel, emollient, or soap.

25. The method of claim 16, wherein the subject is a mammal.

26. The method of claim 25, wherein the mammal is a mouse or rat.

27. The method of claim 25, wherein the mammal is a human.

28. A method for controlling a chronic bacterial infection, comprising administering to a subject in need thereof an effective amount of a composition comprising palmitoleic acid, wherein the amount is effective to prevent, inhibit, reduce, or remove a biofilm.

29. The method of claim 28, wherein the composition further comprises at least one antimicrobial agent.

30. The method of claim 29, wherein the antimicrobial agent is an antibiotic.

31. The method of claim 30, wherein the antibiotic may is selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides.

32. The method of claim 28, wherein the subject is a mammal.

33. The method of claim 32, wherein the mammal is a mouse or rat.

34. The method of claim 32, wherein the mammal is a human.

35. The method of claim 28, wherein the chronic bacterial infection is selected from the group consisting of urinary tract infection, gastritis, lung infection, ear infection, cystitis, pyelonephritis, arterial damage, leprosy, tuberculosis, benign prostatic hyperplasia, chronic prostatitis, osteomyelitis, bloodstream infection, skin infection, rosacea, open wound infection, chronic wound infection, and sinus infection.

36. The method of claim 28, wherein the chronic bacterial infection results from an infection of bacteria selected from the group consisting of Streptococcus pyogenes, Streptococcus pneumoniae, Escherichia coli, Pseudomonas aeruginosa, Staphylococcus aureus, Staphylococcus epidermidis, Propionibacterium acnes, and Haemophilus influenzae.

37. A method for identifying an agent capable of preventing, reducing, inhibiting or removing a biofilm, comprising
a. contacting a biofilm with the agent, wherein the agent is a derivative of a palmitoleic acid;
b. quantifying the biofilm before and after contacting the biofilm with the agent;
c. selecting the agent that prevents, reduces, inhibits or removes at least about 50% of the biofilm, wherein the agent does not inhibit bacterial growth when the concentration amount of the agent is at least twice the amount needed to prevent, reduce, inhibit, or remove at least 50% of the biofilm.

38. The method of claim 37, wherein in step b) the biofilm is stained with a dye and measured using a spectrophotometer to obtain the quantitative value for the mass of each biofilm.

39. A pharmaceutical composition for reducing, preventing, inhibiting or removing biofilm formation comprising a pharmaceutically acceptable carrier or diluent and a therapeutically effective amount of 0.01 to 50% of palmitoleic acid.

40. The pharmaceutical composition of claim 39, further comprising at least one antimicrobial agent.

41. The pharmaceutical composition of claim 40, wherein the antimicrobial agent is an antibiotic.

42. The pharmaceutical composition of claim 41, wherein the antibiotic may is selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides.

43. An animal food composition comprising palmitoleic acid, wherein the animal food composition promotes animal dental hygiene by preventing, reducing, inhibiting or removing a biofilm.

44. The animal food composition of claim 43, wherein the animal food composition is a dry or semi-moist pet food composition.

45. The animal food composition of claim 44, wherein the pet food composition is a pet biscuit.

46. The animal food composition of claim 43, wherein the pet food composition is a dog or cat food.

47. The animal food composition of claim 43, wherein the pet food composition is applied to the surface of a chewable pet toy.

48. The animal food composition of claim 43, wherein the biofilm is a dental plaque.

49. A dentifrice composition comprising palmitoleic acid, wherein the dentifrice composition promotes dental hygiene by preventing, reducing, inhibiting or removing a biofilm.

50. The dentifrice composition of claim 49, wherein the dentifrice composition is selected from the group consisting of toothpaste, toothpowder, liquid dentifrice, mouth detergent, mouthwash, troches, chewing gum, dental or gingival massage cream, dental strip, dental gel, and gargle tablet.

51. The dentifrice composition of claim 49, wherein the dentifrice composition comprises about 0.1% to about 20% of palmitoleic acid.

52. The dentifrice composition of claim 51, wherein the dentifrice composition comprises about 2% of palmitoleic acid.

53. The dentifrice composition of claim 49, further comprising at least one antimicrobial agent.

54. The dentifrice composition of claim 53, wherein the antimicrobial agent is an antibiotic.

55. The dentifrice composition of claim 54, wherein the antibiotic may is selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides.

56. A nebulizer for administration of a composition to a subject, wherein the composition comprises palmitoleic acid and wherein the composition prevents, reduces, inhibits or removes a biofilm.

57. The nebulizer of claim 56, wherein the composition comprises about 0.1% to about 5.0% palmitoleic acid.

58. The nebulizer of claim 56, wherein the composition prevents, reduces, inhibits or removes a biofilm in the lungs of the subject.
